(19) United States
(12) Patent Application Publication
(10) Pub. No.: US 2018/0168200 A1
(43) Pub. Date: Jun. 21, 2018

(54) MICROWAVE-ASSISTED STERILIZATION AND PASTEURIZATION OF LIQUID AND SEMI-LIQUID MATERIALS

(71) Applicant: 915 Labs, LLC, Centennial, CO (US)

(72) Inventors: Anthony Robert Knoerzer, Florissant, CO (US); Matthew Raider, Denver, CO (US); David Behringer, Denver, CO (US)

(21) Appl. No.: 15/845,196

(22) Filed: Dec. 18, 2017

Related U.S. Application Data
(60) Provisional application No. 62/436,217, filed on Dec. 19, 2016.

(51) Int. Cl. A23L 3/01; A23L 3/04; A23L 3/00; A61L 2/00

(52) U.S. Cl. CPC A23L 3/01; A23L 3/04; A61L 2/0023; A61L 2/0064; A23L 3/001

(57) ABSTRACT

Methods for pasteurizing or sterilizing liquid or semi-liquid materials in a liquid-filled microwave heating system are described herein. The methods described herein may be used for pasteurizing or sterilizing a variety of different liquids and semi-liquids, including foodstuffs and beverages, as well as medical, pharmaceutical, nutraceutical, and veterinary liquids. Efficient and rapid heating using microwave energy can provide better temperature control, which permits the use of thinner bottles without sacrificing product quality or safety.

### CROSS-REFERENCE TO RELATED APPLICATIONS

This application claims priority to U.S. Provisional Patent Application No. 62/436,217, filed on Dec. 19, 2016, the entire disclosure of which is incorporated herein by reference.

### FIELD OF THE INVENTION

The present invention relates to methods and systems for the microwave-assisted pasteurization and sterilization of liquid and semi-liquid materials in sealed containers.

### BACKGROUND

In recent times, consumer preference has begun shifting toward more health-conscious beverages, such as teas, juices, and isotonics, many of which require hot filling in order to achieve desired shelf-life. However, the temperatures required for hot filling exceed the glass transition temperature of the polymers used to form the beverage containers. To prevent melting, the bottles and other containers used for hot-filled beverages are, on average, 20 to 35 percent thicker than those used for beverages that are not hot filled. Additionally, bottles used for hot filled beverages often include thermal expansion panels, which further increase the thickness of the bottle, while also limiting product design options and consumer appeal.

Thus, a need exists for methods and systems for processing isotonic beverages and other similar liquid and semi-liquid consumable items, that permits the use of thinner containers, while still providing the desired degree of pasteurization or sterilization needed to achieve a suitable shelf-life.

### SUMMARY

One embodiment of the present invention concerns a process for pasteurization or sterilization of a bottled liquid using a microwave heating system. The process comprises (a) providing a plurality of sealed bottles at least partially filled with a liquid, wherein the pressure within each of the sealed bottles is not more than 1.5 atm; (b) passing the at least partially filled bottles through a microwave heating chamber; and (c) during at least a portion of the passing, heating the bottles, wherein at least a portion of the heating is performed using microwave energy. Each of the bottles is formed from a polymeric material and the ratio of the dry empty weight of an individual bottle measured without a cap to the volume capacity for liquid in the individual bottle is not more than 0.040 g/mL.

Another embodiment of the present invention concerns a packaged liquid item. The packaged liquid item comprises a bottle presenting an opening and defining an internal volume, a cap sealing the opening, and a liquid at least partially filling the internal volume of the bottle. The liquid has a total sugar content of at least 1° Brix and the pressure within the sealed bottle is not more than 1.5 atm. The bottle is formed from a polymeric material, and the ratio of the dry empty weight of the bottle measured without the cap to the nominal liquid capacity of the bottle is not more than 0.040 g/mL.

Yet another embodiment of the present invention concerns a process for pasteurization or sterilization of a liquid or semi-liquid material. The process comprises (a) introducing a plurality of a bottles into a microwave heating chamber, wherein each of the bottles are at least partially filled with the liquid or semi-liquid material, wherein the ratio of the maximum length of each of the bottles to its maximum diameter is at least 2:1; (b) passing the bottles into a heating zone, wherein the heating zone is at least partially filled with a liquid medium; and (c) heating the bottles in the heating zone, wherein at least a portion of the heating is performed using microwave energy. The bottles are submerged in the liquid medium during the heating, and each of the bottles has a residence time within the heating zone that is within about 10 percent of the residence time of each of the other bottles heated in the heating zone.

Still another embodiment of the present invention concerns a process for the pasteurization or sterilization of bottled water using a microwave heating system. The process comprises (a) at least partially filling a plurality of bottles with water; (b) sealing the at least partially filled bottles of water with at least one sealing device; (c) passing the sealed bottles of water through a microwave heating chamber; (d) continuously directing microwave energy toward the bottles of water passing through the microwave heating chamber; and (e) heating the bottles of water to a target temperature sufficient to pasteurize or sterilize the water within the bottles using at least a portion of the microwave energy.

A further embodiment of the present invention concerns a process for pasteurization or sterilization of liquid or semi-liquid material. The process comprises (a) passing a plurality of containers at least partially filled with the liquid or semi-liquid material through a microwave heating chamber at least partially filled with a liquid medium, wherein the containers are at least partially formed from a polymeric material having a glass transition temperature; (b) discharging microwave energy into the microwave heating chamber via at least one microwave launcher; and (c) heating the containers using at least a portion of the microwave energy discharged into the microwave heating chamber. The containers are submerged in the liquid medium during the heating. The heating is sufficient to increase the minimum temperature of the coldest region of the liquid or semi-liquid medium to temperature at or above a target temperature for a predetermined amount of time, and the target temperature is greater than the glass transition temperature of the polymeric material. During at least a portion of the heating, the average temperature of the liquid medium at the wall of each of the containers is below the glass transition temperature of the polymeric material.

A still further embodiment of the present invention concerns a process for pasteurization or sterilization of liquid or semi-liquid material. The process comprises (a) at least partially filling a plurality of containers with the liquid or semi-liquid material, wherein the maximum temperature of the liquid or semi-liquid material during the filling is in the range of from 110° F. to a first target temperature; (b) passing the containers through a microwave heating zone on a convey line; (c) continuously directing microwave energy toward the containers passing through the microwave heating zone on the convey line; and (d) heating the containers to a second temperature that is greater than the first target temperature using at least a portion of the microwave energy in order to pasteurize or sterilize the liquid or semi-liquid material within each of the containers.

An even further embodiment of the present invention concerns a microwave heating system for heating a plurality of containers filled with a liquid or semi-liquid material. The system comprises at least one microwave generator for generating microwave energy, a microwave heating chamber for heating the containers, wherein the microwave heating chamber has an inlet and an outlet, and at least two spaced apart microwave launchers for emitting at least a portion of the microwave energy into the microwave heating chamber, wherein at least a portion of the microwave energy is used to heat the containers. The inlet to the microwave heating chamber is at a higher vertical elevation than the outlet of the microwave heating chamber.

### BRIEF DESCRIPTION OF THE DRAWINGS

Various embodiments of the present invention are described in detail below with reference to the attached drawing Figures, wherein:

FIG. 1a is a schematic depiction of the major steps of a method for microwave pasteurizing or sterilizing liquid or semi-liquid materials in bottles or other containers according to embodiments of the present invention;

FIG. 1b is a schematic depiction of the major zones of a system for microwave pasteurizing or sterilizing liquid or semi-liquid materials in bottles or other containers according to embodiments of the present invention;

FIG. 2a is a perspective view of a carrier for transporting a plurality of bottles or other containers through a microwave pasteurization or sterilization system according to embodiments of the present invention;

FIG. 2b is an end view of the carrier shown in FIG. 2a;

FIG. 2c is a side view of the carrier shown in FIGS. 2a and 2b;

FIG. 3 is a perspective view of another carrier for transporting a plurality of bottles or other containers through a microwave pasteurization or sterilization system according to embodiments of the present invention;

FIG. 4a is a side view of bottles passing through a tunnel in a single-file line, particularly illustrating one example of a side-by-side configuration;

FIG. 4b is a side view of bottles passing through a tunnel in a single-file line, particularly illustrating one example of an end-to-end configuration;

FIG. 5 is a schematic partial side cut-away view of a microwave heating zone configured according to embodiments of the present invention, particularly illustrating the arrangement of the microwave generator, microwave heating chamber, and microwave distribution system within the microwave heating zone;

FIG. 6 is a schematic partial side cut-away view of a microwave heating zone configured according to embodiments of the present invention, particularly illustrating the arrangement of bottles in multiple tunnels arranged in the microwave heating chamber;

FIG. 7a is a schematic partial side cut-away view of a microwave heating zone configured according to embodiments of the present invention, particularly illustrating an example of a vertical spiral microwave heating chamber;

FIG. 7b is a schematic partial side cut-away view of a microwave heating zone configured according to embodiments of the present invention, particularly illustrating an example of a horizontal spiral microwave heating chamber;

FIG. 8 is a graphical depiction of the temperature of the liquid or semi-liquid material in a bottle or other container processed according to embodiments of the present invention as a function of time and position in the microwave heating zone; and

FIG. 9 is a schematic diagram of the major steps of a process for sterilizing bottled water according to embodiments of the present invention.

### DETAILED DESCRIPTION

The present invention generally relates to methods and systems capable of rapidly and uniformly pasteurizing, sterilizing, or pasteurizing and sterilizing a liquid or semi-liquid material in a bottle or other container without exposing the container or its contents to the harsh operating conditions that are common in traditional processes. The processes and system of the present invention may also reduce, or eliminate, the need for pretreatment steps such as hot filling and aseptic processing, while still providing liquid and semi-liquid products having the requisite degree of pasteurization or sterilization. At the same time, because the container and its contents are exposed to less severe operating conditions, the containers used to hold the liquid or semi-liquid may be between 10 and 50 percent thinner than conventional containers, which results in reduced operating and raw material costs. Furthermore, the liquid or semi-liquid being treated is not overheated or overcooked during processing, which permits a higher quality end product having desirable organoleptic properties, such as taste, texture, and color. Additionally, many of the liquids and semi-liquids processed as described herein may be shelf-stable, which may lead to a significant reduction in refrigeration requirements across the supply chain resulting in even more cost savings.

Embodiments of the present invention may be carried out in a variety of different microwave heating systems including, for example, those similar to the microwave heating systems described in U.S. Patent Application Publication No. US 2013/0240516 (“the '516 application”), which is incorporated herein by reference to the extent not inconsistent with the present disclosure. In addition, embodiments of the present invention can be carried out in the microwave heating system described in U.S. Pat. No. 7,119,313.

In general, pasteurization involves the rapid heating of a liquid or semi-liquid to a minimum temperature between 80° C. and 100° C., while sterilization involves heating the liquid or semi-liquid to a minimum temperature between about 100° C. and about 140° C. However, because pasteurization and sterilization may take place simultaneously, or nearly simultaneously, the processes and systems described herein may be configured for pasteurization, sterilization, or both pasteurization and sterilization. Processes and systems as described herein may be configured to pasteurize, sterilize, or pasteurize and sterilize a plurality of bottles or other types of containers, a liquid or semi-liquid material enclosed therein, or both.

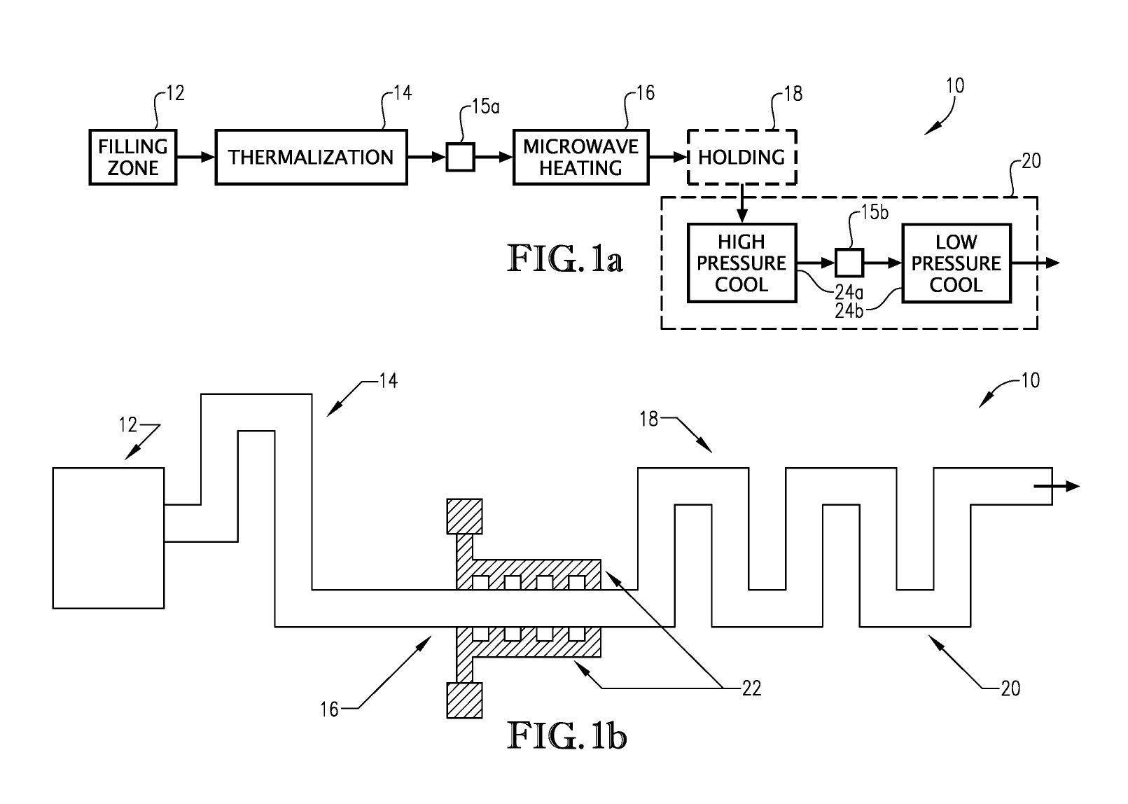

Turning initially to FIGS. 1a and 1b, schematic diagrams of the main steps of a microwave heating process and the main elements of a microwave heating system 10 suitable for use according to embodiments of the present invention are provided. As shown in FIGS. 1a and 1b, the microwave heating system includes a filling zone 12, a thermalization zone 14, a microwave heating zone 16, a hold zone 18, a quench zone 20, and optional transfer zones 15a,b. Bottles or other containers may be introduced from the filling zone 12 into the thermalization zone 14, wherein the bottles are heated to a substantially uniform temperature. Once preheated in the thermalization zone 14, the bottles or other containers may be passed through a transfer zone 15a before being introduced into the microwave heating zone 16. In the microwave heating zone 16, the bottles or other containers may be rapidly heated using microwave energy discharged into the microwave heating zone 16 by one or more microwave launchers 22 shown in FIG. 1b. The heated containers may optionally be passed through a hold zone 18, wherein the bottles are permitted to thermally equilibrate so that the coldest portion of the liquid or semi-liquid contents are maintained at a temperature at or above a predetermined target temperature (e.g., a pasteurization or sterilization target temperature) for a specified amount of time.

Subsequently, the bottles may then be passed to a quench zone 20, wherein the bottles may be cooled to a suitable handling temperature. In some cases, the quench zone 20 may be divided into a high-pressure cooling zone 24a and a low-pressure cooling zone 24b and can include another transfer zone 15b between the two cooling zones 24a,b. Alternatively, the quench zone 20 may include a single cooling zone with transfer zone 15b located upstream or downstream of the cooling zone (not shown). As used herein, the term “upstream” and “downstream” refer to the relative positions of various components or zones along the main flow path through the microwave heating system. A component or zone located prior to another can be said to be “upstream” of that component, while a component or zone located after another may be said to be “downstream” of that component.

In some cases, two or more of the thermalization zone 14, microwave heating zone 16, hold zone 18, and quench zone 20 may be defined within a single vessel, while, in other cases, at least one of these zones may be defined within one or more separate vessels. Additionally, in some cases, one or more of the vessels may be configured to be at least partially filled with a liquid medium in which the bottles being processed can be at least partially submerged during processing. As used herein, the term “at least partially filled” means at least 50 percent of the volume of the specified vessel is filled with a liquid medium. In some cases, the volume of at least one of the vessels used in the thermalization zone 14, microwave heating zone 16, hold zone 18, and quench zone 20 can be at least about 75 percent, at least about 90 percent, at least about 95 percent, or nearly 100 percent filled with a liquid medium.

When present, the liquid medium used may include any suitable type of liquid. In some cases, the liquid medium may have a dielectric constant greater than the dielectric constant of air and/or it may have a dielectric constant similar to the dielectric constant of the liquid or semi-liquid within the bottles being processed. Water (or a liquid medium comprising water) may be particularly suitable. The liquid medium may also include one or more additives, such as, for example, oils, alcohols, glycols, and salts, to alter or enhance its physical properties (e.g., boiling point) of the liquid medium at the conditions of operation of the system.

Additionally, the microwave heating system 10 may include a conveyance system (not shown in FIGS. 1a and 1b) comprising one or more convey segments for transporting the bottles through one or more of the processing sections described above. Examples of suitable types of convey segments include, but are not limited to, plastic or rubber belt conveyors, chain conveyors, roller conveyors, flexible or multi-flexing conveyors, wire mesh conveyors, bucket conveyors, pneumatic conveyors, screw conveyors, trough or vibrating conveyors, and combinations thereof. In some cases, the conveyance system can include one or more tunnels extending through one or more of the zones through which the bottles may be passed. Any suitable number of individual convey segments (or tunnels) can be used with the conveyance system, and the convey segment or segments may be arranged in any suitable manner within the vessels. Other examples of convey systems suitable for use in the present invention are described in the '516 application.

Turning again to FIGS. 1a and 1b, the microwave heating system 10 of the present invention may be configured to process a plurality of containers at least partially filled with a liquid or semi-liquid material. As used herein, the term “liquid” refers to a material that exists in a liquid state at ambient temperature and pressure, while the term “semi-liquid” refers to a material including a liquid phase or portion, but that also includes, or exhibits properties, similar to solids and/or gases under ambient conditions. Examples of semi-liquids may include, but are not limited to, emulsions, dispersions, colloids, suspensions, pastes, and gels. Liquids and semi-liquids that may be processed according to embodiments of the present invention may comprise foodstuffs, beverages, medical fluids, pharmaceutical fluids, nutraceutical fluids, or veterinary fluids, although other liquids and semi-liquids may also be suitable.

Although processes and systems of the present invention are described herein with respect to processing “containers,” it should be understood that this term is not limited to a particular package shape or configuration, but instead broadly encompasses any item capable of holding some volume of liquid or semi-liquid material. Examples of suitable types of containers can include, but are not limited to, bottles, trays, jugs, cartons, bags, pouches, tubes, and tubs. Containers suitable for use in the present invention may range in size, and can have, for example, a nominal liquid capacity as low as 1 fluid ounce (fl. oz.) or as high as 150 fl. oz. or more. As used herein, the term “nominal liquid capacity” refers to the volume of liquid or semi-liquid material that the container is designed to hold, while the term “maximum liquid capacity” refers to the maximum possible volume of liquid that a given container is capable of holding.

Containers suitable for use with the present invention can have a nominal liquid capacity of at least about 1.5, at least about 2, at least about 4, at least about 6, at least about 8, at least about 10, at least about 12, at least about 15, at least about 18, at least about 20, at least about 22, at least about 24, at least about 28, at least about 30, at least about 32, at least about 34, at least about 36, at least about 38, at least about 40, at least about 42, at least about 48, at least about 52, or at least about 56 fl. oz., and/or not more than about 150, not more than about 140, not more than about 130, not more than about 128, not more than about 122, not more than about 120, not more than about 118, not more than about 112, not more than about 110, not more than about 106, not more than about 100, not more than about 96, not more than about 90, not more than about 86, not more than about 80, not more than about 76, not more than about 70, or not more than about 64 fl. oz. In some cases, containers used in the present invention can have a nominal liquid capacity in the range of from about 4 to about 40 fl. oz., about 6 to 38 fl. oz., or about 8 to 36 fl. oz.

The containers processable as described herein may have a variety of shapes including, but not limited to, cubic, cylindrical, prism, or polygonal. Each container can have a length (longest dimension) of at least about 2 inches, at least about 4 inches, at least about 6 inches and/or not more than about 18 inches, not more than about 12 inches, or not more than about 10 inches; a width (second longest dimension) of at least about 1 inch, at least about 2 inches, at least about 4 inches and/or not more than about 12 inches, not more than about 10 inches, or not more than about 8 inches; and/or a depth (shortest dimension) of at least about 0.5 inches, at least about 1 inch, at least about 2 inches and/or not more than about 8 inches, not more than about 6 inches, or not more than about 4 inches. In some applications, the containers can comprise individual items or packages having a generally rectangular or prism-like shape, while, in other applications, the containers may comprise elongated bottles having a ratio of the maximum length to maximum diameter of at least 2:1, at least about 2.5:1, or at least about 3:1. Other shapes are possible and should be considered to fall within the scope of the present invention.

Additionally, the containers may be formed of a wide variety of suitable materials. In some cases, the containers may be at least partially formed from a polymeric material having, for example, a glass transition temperature of at least about 50, at least about 55, at least about 60, at least about 65, at least about 70, at least about 75, at least about 80, or at least about 85° C. Examples of suitable polymeric materials can include, but are not limited to, polyethylene terephthalate, polyethylene, polypropylene, polyamide, EVOH, polylactic acid, polyhydroxyalkanoates, polybutylene succinate, biopolymers, and combinations thereof. Containers formed from non-polymeric materials, such as glass or other at least partially microwave-transparent materials, may also be used in various embodiments in the present invention. In some cases, the containers may be formed of virgin materials, while, in others at least about 5, at least about 10, at least about 15, at least about 20, at least about 25, at least about 30, at least about 35, at least about 40, at least about 45, at least about 50, at least about 55, at least about 60, at least about 65, at least about 70, or at least about 75 percent or more of the container may be formed from a recycled, recyclable, or compostable material.

It has been discovered that processes and systems as described herein may be particularly useful for the pasteurization or sterilization (or both) of microbiologically susceptible liquids and semi-liquids such as, for example, isotonic beverages, as well as teas, juices, and a variety of other food, beverage, pharmaceutical, medical, nutraceutical, and veterinary liquid and semi-liquid materials and for the purification of water. Microbiologically susceptible liquids and semi-liquids have various properties conducive to the growth of bacteria, mold, fungus, and other microbiological contaminants. For example, in some cases, microbiologically susceptible liquids and semi-liquids may have a non-acidic pH, may not be pressurized or carbonated, may have a high sugar content, and/or a low preservative content, although not all of these are required.

The properties of the liquid or semi-liquid material processed according to the present invention may vary widely. In general, the pH of the liquid or semi-liquid material can range from 0 to 13. In some cases, the pH can be at least about 3, at least about 3.5, at least about 4, at least about 4.5, at least about 5, at least about 5.5, at least about 6, or at least about 6.5, or higher, while, in other cases, the pH can be less than 3, not more than about 2.5, not more than about 2, not more than about 1.5, or not more than about 1. Additionally, the liquid or semi-liquid material could have a variety of viscosities and can be, for example, a liquid, or even a gel or paste.

The sugar content of the liquid or semi-liquid material, measured by the Brix scale, can be at least about 1, at least about 1.5, at least about 2, at least about 2.5, at least about 3, or at least about 3.5°, or it can be less than 2, less than 1.5, less than 1, or even 0° Brix. In some cases, the sugar content of the liquid or semi-liquid material can be at least about 2, at least about 2.5, at least about 3, at least about 3.5, at least about 4, at least about 4.5, or at least about 5 and/or not more than about 20, not more than about 18, not more than about 15, not more than about 12, not more than about 10, or not more than about 8 grams of sugar per milliliter of solution (g/mL).

In some applications, the liquid or semi-liquid material can have a preservative content of not more than about 3, not more than about 2.5, not more than about 2, not more than about 1.5, or not more than about 1 weight percent, or it may include higher concentrations of one or more preservatives. Examples of preservatives can include, but are not limited to, benzoic acid, sorbic acid, salts thereof, and combinations thereof. In some applications, the liquid or semi-liquid material may be nutritive or non-nutritive and can include pulp or particles having an average size of not more than 30, not more than about 25, not more than about 20, not more than about 15, not more than about 10, not more than about 5, not more than about 2, or not more than about 1 mm, or in the range of 1 to 30 mm, 2 to 27 mm, or 5 to 25 mm.

Optionally, the liquid or semi-liquid material may include one or more additives selected from the group consisting of natural or synthetic sweeteners such as sucrose, fructose, and high fructose corn syrup, acidification agents such as phosphoric and/or citric acid, colors, foaming agents, emulsifying agents, vitamins, electrolytes, and combinations thereof. The types and/or amounts of each additive may be varied depending on the particular application.

Specific examples of suitable liquid or semi-liquid materials can include, but are not limited to, beverages such as teas, juices, mineral drinks, electrolyte drinks, energy drinks, vitamin drinks, shakes, smoothies, dairy drinks, alcoholic drinks, and coffee drinks. Other suitable beverages can include shelf-stable dairy drinks, including, but not limited to milk and cream, as well as carbonated soft drinks. Examples of suitable liquid or semi-liquid material foods can include, but are not limited to, jams, jellies, soups, stews, sauces, salsas, creams such as whipped cream, condiments such as ketchup, mustard, mayonnaise, salsas, and syrups. In some applications, the food or beverage may be pressurized or carbonated, while, in other applications, the food or beverage may not be. Additionally, the liquid or semi-liquid material may also be a medical fluid such as, for example, sterile eye wash, and other sterile liquids or medicines. In some case, the liquid may be or comprise water.

Conventionally, many liquids and semi-liquids and, in particular, microbiologically susceptible liquid or semi-liquid material, are processed by hot filling or aseptically processing the material or its container before or during the fill step in order to achieve the requisite kill rate and/or desired shelf life for the final product. Examples of suitable kill rates can be in the range of 1 log to 8 log kill, in the range of 2 log to 7 log kill, or in the range of 3 log to 6 log kill. One drawback of these conventional processes is that the temperatures required for the hot filling and aseptic processing steps often exceed the glass transition temperature of the polymeric material from which the container is formed. As result, bottles and other containers used in hot filling and/or aseptic processes are often excessively thick and include design features, such as expansion panels, that are required to prevent deformation or damage to the container. For example, conventional bottles for isotonic beverages formed from polyethylene terephthalate (PET) typically have a ratio of the dry empty weight of an individual bottle measured without the cap to the nominal liquid capacity of the bottle greater than 0.050 g/mL.

However, it has been discovered that pasteurizing or sterilizing liquids and semi-liquids according to embodiments of the present invention may eliminate the need for hot filling, aseptic processing, and other such pretreatment steps. As a result, the thickness of the container being used may be reduced, thereby providing a substantial reduction of energy and raw material costs. Additionally, features such as expansion panels may be eliminated, increasing the appeal while also reducing the cost of the container. It has been found, for example, that polymeric bottles used for holding liquids having a total sugar content of at least 1° Brix and a sealed pressure of less than 1.5 atm can be not more than 0.040, not more than about 0.037, not more than about 0.035, not more than about 0.032, or not more than about 0.030 g/mL, which represents an overall reduction of 10 to 50 percent as compared to conventional hot-fill containers.

Turning back to FIGS. 1a and 1b, in some embodiments, the microwave heating system 10 may include a filling zone 12 for at least partially filling the containers with a liquid or semi-liquid material. In some applications, the target filling temperature of the liquid or semi-liquid material introduced into the containers in the filling zone 12 shown in FIGS. 1a and 1b, may be in the range of from at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, or at least about 120° F. to a target temperature of less than about 185, not more than about 180, not more than about 175, not more than about 170, not more than about 165, not more than about 160, not more than about 155, not more than about 150, not more than about 145, not more than about 140, not more than about 135, not more than about 130, or not more than about 125° F.

Typically, the target temperature may be lower than the conventional hot-fill temperature for a given liquid or semi-liquid material. In some applications when the containers being filled are formed from a polymeric material, the target filling temperature may be less than the glass transition temperature of the polymeric material and it can, in some cases, be at least about 2, at least about 5, at least about 8, at least about 10, or at least about 12° F. less than the glass transition temperature of the polymer material. This is in contrast to traditional hot-filling processes, which may introduce liquid or semi-liquid material into containers at or just above the glass transition temperature of a polymeric container.

In some cases, the liquid or semi-liquid material may be introduced into the containers at a relatively cool temperature of less than, for example, 80° F., and the filled bottles may then be preheated before or after being sealed. In other applications, the liquid or semi-liquid material may be introduced into the containers at a warm temperature between, for example, about 95, about 100, about 105, or about 110° F. and the target filling temperature. In such cases, the filled bottles may optionally be preheated in the filling zone before or after being sealed, and/or may be additionally heated to achieve a substantially uniform temperature in the thermalization zone 14, as shown in FIGS. 1a and 1b.

As discussed previously, in some cases, the systems and processes of the present invention may reduce or eliminate the need for common pre-treatment steps such as, for example, hot filling, aseptic processing, reverse osmosis, and other similar processes. In some applications, the liquids and semi-liquids previously required to be hot-filled or aseptically processed may be directly introduced into containers in the present invention without these steps being performed. As a result, the containers and liquid or semi-liquid material are not pasteurized or sterilized prior to the filling step, and the liquid or semi-liquid may be introduced into the container at a lower temperature within one or more of the ranges described herein.

Before exiting the filling zone, the at least partially filled containers may be sealed with a sealing device to provide sealed containers suitable for introduction into the thermalization and/or microwave heating zones. The specific type of sealing device may depend on the particular type of container and can include, for example, caps or lids having a variety of different closures (e.g., snap-on, screw-on, etc.). The containers may require any number of sealing devices, and, in some cases, may not require any sealing device when, for example, the container is a single-use or tamper-evident container. The sealed containers may be pressurized or unpressurized, depending on the specific application. In some applications, the internal pressure of the sealed container can be greater than 3, at least about 3.25, at least about 3.5, or at least about 4 atm, while, in other applications, it may not more than about 3, not more than about 2.75, not more than about 2.5, not more than about 2.25, not more than about 2, not more than about 1.75, or not more than about 1.5 atm. When pressurized, the liquid or semi-liquid material may be pressurized with any suitable gas including, but not limited to, nitrogen, carbon dioxide, and combinations thereof. The containers can be filled and sealed using any suitable type of device or system including, but not limited to a rotary filler.

In other embodiments, the microwave system 10 may not include a filling zone and the filled bottles or other containers being pasteurized or sterilized may be introduced directly into the thermalization zone 14. In such embodiments, the bottles or other containers may be filled at another location by another party, and transported to the microwave heating system to be pasteurized or sterilized as described herein.

As shown in FIGS. 1a and 1b, sealed containers exiting the filling zone 12 may be transferred into an optional thermalization zone 14. When present, the thermalization zone 14 may use thermal energy to heat the containers to a substantially uniform temperature. In some applications, at least about 85, at least about 90, at least about 95, at least about 97, or at least about 99 percent of all the containers withdrawn from the thermalization zone have a temperature within about 10, within about 5, within about 2, within about 1° C. of one another. As used herein, the terms “thermalize” and “thermalization” generally refer to a step of temperature equilibration or equalization. In some cases, the temperature of the coldest portion of the liquid or semi-liquid in the bottles introduced into the thermalization 14 zone can be not more than about 45, not more than about 40, not more than about 35, not more than about 30, not more than about 27, or not more than about 25° C.

In some applications, the thermalization zone 14 may include a chamber at least partially filled with a liquid medium, such that the containers are submerged in and pass through the liquid during the thermalization step. Optionally, the thermalization chamber may be operated under pressure, such that the containers are exposed to a pressure that is at least about 2, at least about 5, at least about 7, at least about 10, or at least about 15 psi greater than the pressure of the fluid surrounding the containers at ambient conditions. In some embodiments, at least a portion of the thermalization zone 14 may be operated within about 10, within about 8, within about 5, within about 2 or at ambient pressure. In other applications, the pressure in the thermalization chamber may only be due to the surrounding fluid pressure. In some cases, the system 10 may not include a thermalization zone such that the containers are directly introduced into the microwave heating zone 16 from the filling zone 12.

When pressurized, the thermalization step may be performed at a pressure of at least about 5, or at least about 10 psig and/or not more than about 80, not more than about 50, not more than about 40, or not more than about 25 psig. The containers passing through the thermalization zone 14 can have an average residence time of at least about 1 minute, at least about 5 minutes, at least about 10 minutes and/or not more than about 60 minutes, not more than about 20 minutes, or not more than about 10 minutes. The liquid or semi-liquid in the containers withdrawn from the thermalization zone 14 can have an average temperature of at least about 20° C., at least about 25° C., at least about 30° C., at least about 35° C. and/or not more than about 90° C., not more than about 75° C., not more than about 60° C., or not more than about 50° C.

In some embodiments, at least one transfer zone 15a may exist to transport the containers from the filling zone 12 to the thermalization zone 14, if present, and, if not present, to the microwave heating zone 16. When present, the transfer zone 15a may include one or more transfer devices for moving the plurality of containers from the filling zone 12 to the thermalization zone 14 or the microwave heating zone 16. Examples of transfer devices can include, but are not limited to, a revolving door, a screw drive, or gate valve. In other applications, the containers may be passed through a vertical column of water or other fluid having a height of at least about 5, at least about 10, at least about 15, at least about 20, at least about 25, or at least about 30 feet in order to transition the containers from ambient pressure to the higher pressure of a liquid-filled thermalization zone 14 or microwave heating zone 16.

While being introduced into the thermalization zone 14 and, if no thermalization zone, into the microwave heating zone 16, the containers may be configured in any suitable arrangement to help facilitate their passage through the microwave heating system 10. In some cases, the containers passing through the thermalization zone 14 and/or the microwave zone 16 can be arranged in such a way as to control the total residence time of each container within each processing zone. For example, in some applications, each of the containers passed through one of the thermalization zone 14 and/or microwave heating zone 16 can have a residence time within that zone that is within about 25, within about 20, within about 15, within about 10, within about 5, or within about 2 percent of the residence times of each of the other containers passed through the same zone. Control of the residence time of the individual containers may help ensure overall product quality, consistency, and safety.

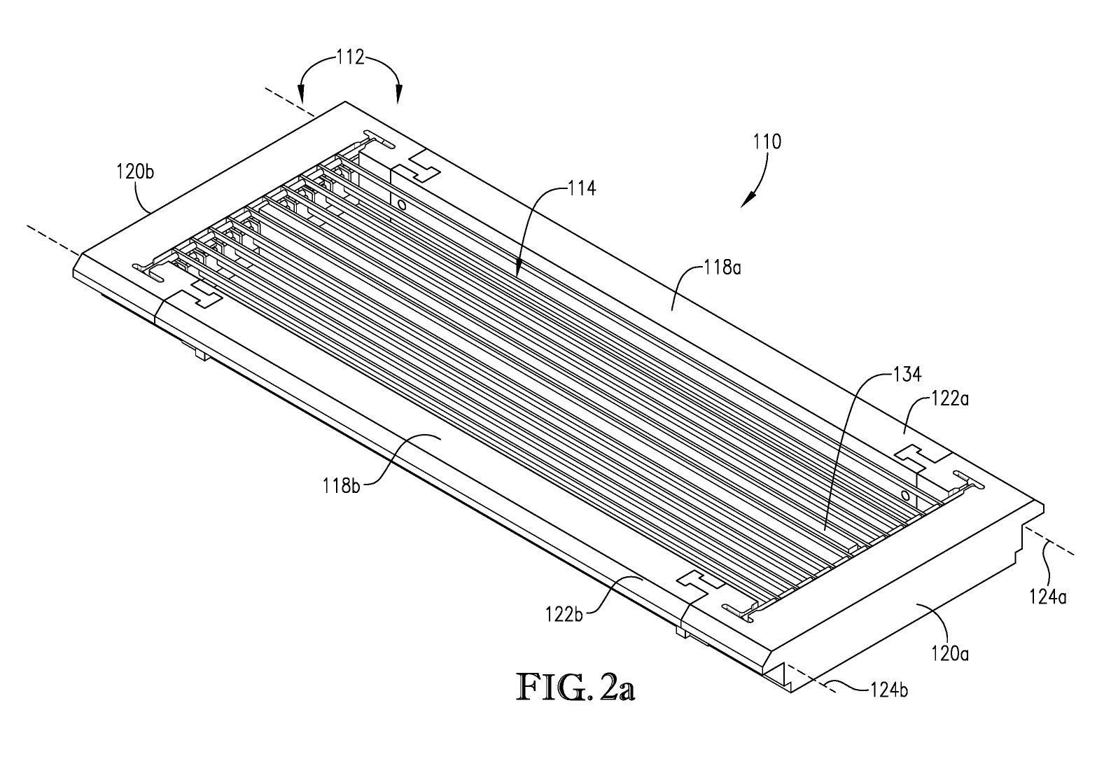

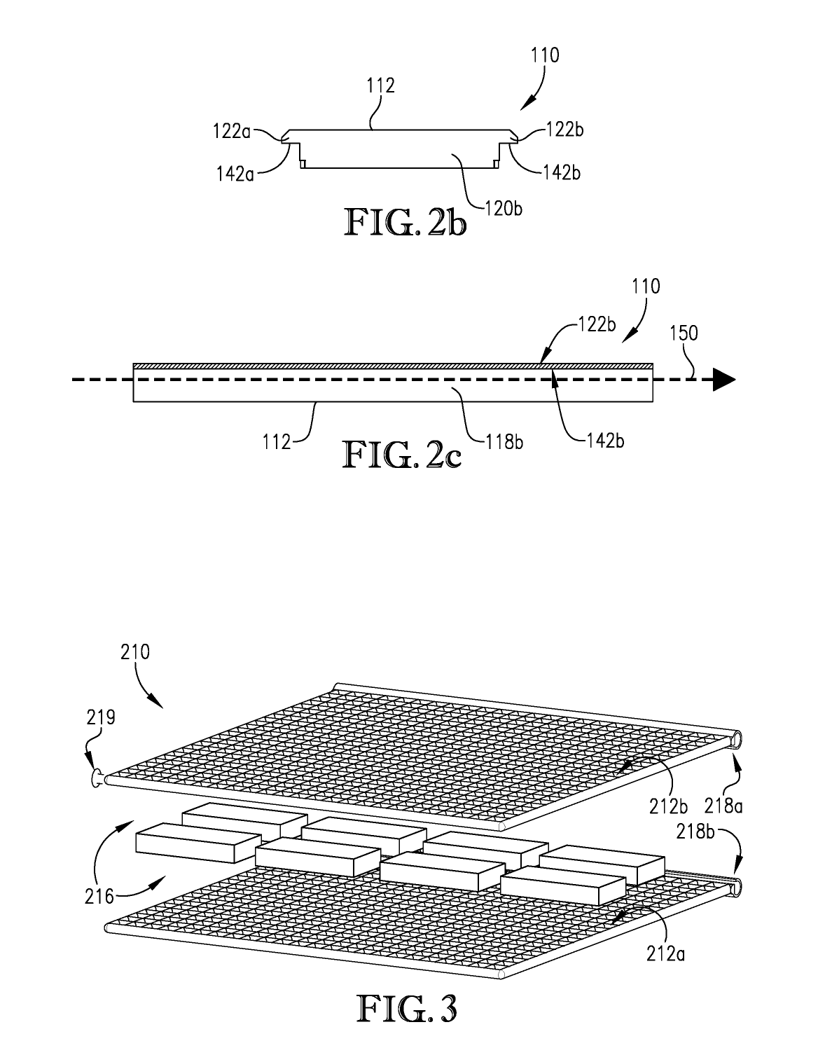

In some embodiments, the residence time of the containers may be controlled by securing the containers in one or more carriers, which are transported through the system via a conveyance system (not shown) including one or more convey lines. One example of a carrier suitable in the microwave heating system according to various embodiments of the present invention is described in U.S. Patent Application Publication No. 2017/0099706, which is incorporated herein by reference to the extent not inconsistent with the present disclosure. Several exemplary views of such a carrier are provided in FIGS. 2a-c. As generally shown in FIGS. 2a-c, the carrier 110 includes an outer frame 112, an upper support structure 114, and a lower support structure 116. The outer frame 112 comprises two spaced-apart side members 118a,b and two spaced-apart end members 120a,b. The first and second end members 120a,b may be coupled to and extend between opposite ends of first and second side members 118a,b to form outer frame 112. When side members 118a,b are longer than the end members 120a,b, the frame 112 may have a generally rectangular shape, as shown in FIGS. 2a-c.

As shown in FIGS. 2a-3c, the first and second side members 118a,b of the carrier 110 each include respective support projections 122a,b that are configured to engage respective first and second convey line support members, which are represented by dashed lines 124a and 124b in FIG. 2b. The first and second support projections 122a,b of carrier 110 present first and second lower support surfaces 142a,b for supporting carrier 110 on first and second convey line support members 124a,b. Convey line support members 124a,b may be a moving convey line element such as, for example, a pair of chains (not shown in FIGS. 2a-c) located on each side of carrier 110 as it moves through the microwave heating system in a direction represented by the arrow in FIG. 2c.

Carriers suitable for use in the microwave heating system described herein may be formed of any suitable materials, including low loss materials, and, in some cases, even electrically conductive materials. For example, carriers suitable for use in microwave heating system 110 can comprise or be constructed of plastic, fiberglass, or any other dielectric material and may be made of one or more microwave-compatible and/or microwave-transparent materials and may be a lossy material. In some embodiments, the carrier can comprise substantially no metal.

In other embodiments, the carrier may include a plurality of support members, shown in FIG. 1a as support members 134, formed from a strong, electrically conductive material. Suitable electrically conductive materials can have a conductivity of at least about 10³ Siemens per meter (S/m), at least about 10⁴ S/m, at least about 10⁵ S/m, at least about 10⁶ S/m, or at least about 10⁷ S/m at 20° C., measured according to ASTM E1004 (09). Additionally, the electrically conductive material may have a tensile strength of at least about 50 MegaPascals (MPa), at least about 100 MPa, at least about 200 MPa, at least about 400 MPa, or at least about 600 MPa, measured according to ASTM E8/E8M-16a, and/or it may also have a yield strength of at least about 50, at least about 100, at least about 200, at least about 300, or at least about 400 MPa at 20° C., measured according to ASTM E8/E8M-16a. The Young's Modulus of the electrically conductive material can be at least about 25 GigaPascals (GPa), at least about 50 GPa, at least about 100 GPa, or at least about 150 GPa and/or not more than about 1000 GPa, not more than about 750 GPa, not more than about 500 GPa, or not more than about 250 GPa, measured at 20° C., measured according to ASTM E111-04 (2010). The electrically conductive material may be metallic and, in some cases, may be a metal alloy. The metal alloy may include any mixture of suitable metal elements including, but not limited to, iron, nickel, and/or chromium. The electrically conductive material may comprise stainless steel and may be food-grade stainless steel.

In some embodiments, the first and second side members 118a,b and first and second end members 120a,b may be formed of any suitable material including, for example, a low loss material having a loss tangent of not more than about 10⁻⁴, not more than about 10⁻³, or not more than about 10⁻², measured at 20° C. Each of the side members 118a,b and end members 120a,b may be formed of the same material, at least one may be formed of a different material. Examples of suitable low loss tangent materials may include, but are not limited to, various polymers and ceramics. In some embodiments, the low loss tangent material may be a food-grade material.

When the low loss material is a polymeric material, it may have a glass transition temperature of at least about 80° C., at least about 100° C., at least about 120° C., or at least about 140° C., so that it may withstand the elevated temperatures to which the carrier may be exposed during heating of the containers. Examples of suitable low loss polymers can include, but are not limited to, polytetrafluoroethylene (PTFE), polysulfone, polynorbornene, polycarbonate (PC), acrylonitrile butadiene styrene (ABS), poly(methyl methacrylate) (PMMA), polyetherimide (PEI), polystyrene, polyvinyl alcohol (PVA), polyvinyl chloride (PVC), and combinations thereof. The polymer can be monolithic or it may be reinforced with glass fibers, such as, for example glass-filed PTFE (“TEFLON”). Ceramics, such as aluminosilicates, may also be used as the low loss material.

Another example of a carrier is illustrated in FIG. 3 as carrier 210. As shown in FIG. 3, carrier 210 comprises a lower securing surface 212a and an upper securing surface 212b configured to secure any suitable number of containers 216 therebetween. In one embodiment, upper and/or lower surfaces 212b and 212a can have a meshed, grid, or grated structure, as generally depicted in FIG. 3, while, in another embodiment, one or both surfaces 212a and 212b can comprise a substantially continuous surface.

Lower and upper securing surfaces 212a and 212b may be attached to one another by a securing device, shown as a fastener 219 in FIG. 3, and, as assembled, carrier 210 may be attached or secured to the conveyance system (not shown) according to any suitable attachment mechanism. In some embodiments, at least one side (or edge) of carrier 210 can include one or more attachment mechanisms, such as, for example, upper and lower hooks 218a and 218b shown in FIG. 2, for securing carrier 210 to a portion (e.g., a bar, a rail, a belt, or a chain) of the conveyance system (not shown). Depending on the thickness and/or weight of containers 216, carrier 210 may only include one of hooks 218a or 218b for securing carrier 210 onto the conveyance system. The conveyance system within microwave heating system 10 shown in FIG. 1 may be configured to transport multiple carriers along one or more conveyance lines and the carriers may be arranged in a side-by-side, laterally-spaced configuration and/or in a vertically-spaced, stacked configuration. When the conveyance system includes a plurality of convey lines, each convey line may include a single carrier for holding a plurality of containers, or each convey line may hold multiple carriers stacked or laterally spaced from each other.

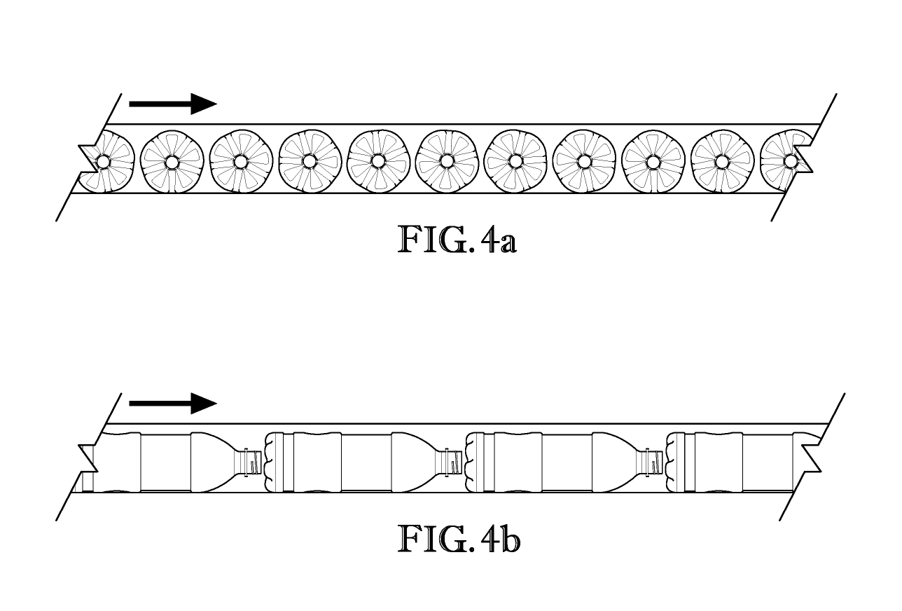

In other applications, the containers may not be secured in a carrier. In such cases, the residence time may be controlled by passing the containers through one or more tunnels sized to permit no more than a single container from passing through at a time. For example, the ratio of the height of the tunnel, measured in a direction perpendicular to the direction of travel of the containers, to the dimension of the containers in the same direction, may be less than 1.5:1, not more than about 1.3:1, not more than about 1.25:1, or not more than about 1.1:1. In some cases, the containers passed through the tunnels may be bottles arranged in a single file line. The bottles may also be arranged in a generally side-by-side configuration, as shown in FIG. 4a or an end-to-end configuration, as generally illustrated in FIG. 4b. Such single file side-by-side and end-to-end configurations could also be used in a carrier or other type of convey line.

As the bottles (or other containers) pass through the thermalization zone 14 and/or microwave heating zone 16 shown in FIGS. 1a and 1b, the bottles may remain in an upright position for all or a portion of the thermalization and/or heating step. In some applications, the bottles or other containers may be inverted before, during, or after being loaded into the thermalization and/or microwave heating zone. Similar configurations are possible with other types of containers and may depend on the particular container and its application.

Referring again to FIGS. 1a and 1b, containers removed from the thermalization zone 14, when present, or, if not present, from the filling zone 12, may be introduced into the microwave heating zone 16, wherein the bottles or other containers may be rapidly heated using microwave energy. As used herein, the term “microwave energy” refers to electromagnetic energy having a frequency between 300 MHz and 30 GHz. Commonly-used industrial frequencies for microwave energy include 915 MHz and 2.45 GHz (2450 MHz). In addition to microwave energy, the microwave heating zone 16 may also employ other types of heating, such as, for example, conductive or convective heating for further increasing the temperature of the bottles passing therethrough. In some cases, at least about 70, at least about 75, at least about 80, at least about 85, at least about 90, or at least about 95 percent of the energy used to heat the bottles within the microwave heating zone 16 can be microwave energy. The microwave energy may heat the bottles directly and/or may be used to heat the fluid surrounding the bottles, which may further heat the bottles by convection and/or conduction.

As the bottles pass through the microwave heating zone 16, the bottles may be heated so that the coldest portion of the contents of each bottle achieves a target temperature. When the microwave heating system is a sterilization or pasteurization system, the target temperature can be a sterilization or pasteurization target temperature of at least about 65° C., at least about 70° C., at least about 75° C., at least about 80° C., at least about 85° C., at least about 90° C., at least about 95° C., at least about 100° C., at least about 105° C., at least about 110° C., at least about 115° C., at least about 120° C., at least about 121° C., at least about 122° C. and/or not more than about 130° C., not more than about 128° C., or not more than about 126° C.

As the bottles or other containers pass through the microwave heating chamber, they may be heated to the target temperature in a relatively short time, which can help minimize any damage or degradation of the liquid or semi-liquid material being heated. For example, the average residence time of each bottle passing through the microwave heating zone can be at least about 5 seconds, at least about 20 seconds, at least about 60 seconds and/or not more than about 10 minutes, not more than about 8 minutes, not more than about 5 minutes, not more than about 3 minutes, not more than about 2 minutes, or not more than about 1 minute. The minimum temperature of the bottles heated in the microwave heating zone can increase by at least about 20° C., at least about 30° C., at least about 40° C., at least about 50° C., at least about 75° C. and/or not more than about 150° C., not more than about 125° C., or not more than about 100° C.

When the microwave heating chamber is liquid-filled, the average bulk temperature of the liquid in the microwave heating chamber may vary and, in some cases, can depend on the amount of microwave energy discharged into the microwave heating chamber. The average bulk temperature of the liquid in the microwave heating chamber can be at least about 70° C., at least about 75° C., at least about 80° C., at least about 85° C., at least about 90° C., at least about 95° C., at least about 100° C., at least about 105° C., at least about 110° C., at least about 115° C., or at least about 120° C. and/or not more than about 135°, not more than about 132° C., not more than about 130° C., not more than about 127° C., or not more than about 125° C. In some cases, this can be at least about 1, at least about 2, at least about 5, at least about 10, at least about 15° C. and/or not more than about 50, not more than about 45, not more than about 40, not more than about 35, not more than about 30, or not more than about 25° C. different (e.g., higher or lower) than the temperature of the contents of the bottle measured at its coldest point.

The microwave heating chamber can be operated at approximately ambient pressure. Alternatively, it may be a pressurized microwave chamber that operates at a pressure that is at least 5 psig, at least about 10 psig, at least about 15 psig, or at least about 17 psig and/or not more than about 80 psig, not more than about 60 psig, not more than about 50 psig, or not more than about 40 psig above ambient pressure. As used herein, the term “ambient” pressure refers to the pressure exerted by the fluid in the microwave heating chamber without the influence of external pressurization devices.

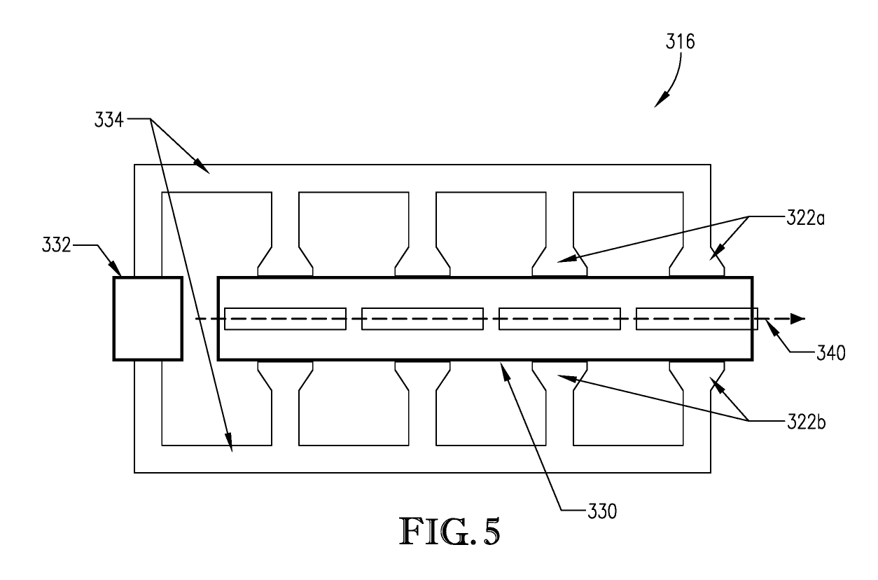

One example of a microwave heating zone 316 configured for use in the microwave heating system described herein is shown schematically in FIG. 5. The microwave heating zone 316 shown in FIG. 5 generally includes a microwave heating chamber 330, at least one microwave generator 332 for generating microwave energy, and a microwave distribution system 334 for directing at least a portion of the microwave energy from the generator (or generators) 332 to the microwave heating chamber 330. The microwave heating zone 316 further comprises one or more microwave launchers 322 for discharging microwave energy from the microwave distribution system 334 into the interior of the microwave heating chamber 330, and a convey system 340 for passing the bottles or carriers loaded with bottles through the microwave heating chamber 330.

The microwave generator 332 can be any suitable device for generating microwave energy of a desired wavelength (λ). Examples of suitable types of microwave generators can include, but are not limited to, magnetrons, klystrons, traveling wave tubes, and gyrotrons. Although illustrated in FIG. 5 as including a single generator, it should be understood that the microwave heating zone may include any number of generators arranged in any suitable configuration. For example, the microwave heating zone can include at least 1, at least 2, at least 3 and/or not more than 5, not more than 4, or not more than 3 microwave generators. Specific configurations of various microwave heating zones including various numbers of generators are discussed in the '516 application.

The microwave distribution system 334 comprises a plurality of waveguides for directing the microwave energy from the generator 332 to the microwave heating chamber 330. The waveguides can be constructed to propagate microwave energy in a specific predominant mode, which may be the same as or different than the mode of microwave energy generated by the generator. As used herein, the term “mode” refers to a generally fixed cross-sectional field pattern of microwave energy. Examples of suitable modes of microwave energy are TE_{xy} mode, wherein x and y are integers in the range of from 0 to 5 and TM_{ab} mode, wherein a and b are integers in the range of from 0 to 5.

The microwave heating zone 316 shown in FIG. 5 further includes at least one microwave launcher for discharging microwave energy into the microwave heating chamber. In the embodiment shown in FIG. 5, the microwave heating zone 316 includes a plurality of upper microwave launchers 322a and a plurality of lower microwave launchers 322b. When the system includes two or more microwave launchers, at least some of the launchers may be positioned on the same side of the microwave heating chamber. These same-side launchers (e.g., upper microwave launchers 322a or lower microwave launchers 322b) may be axially spaced from one another along the length of the microwave heating chamber, in a direction parallel to the direction of travel of the carrier passing through the chamber, as shown in FIG. 5. The microwave system may also include two or more same-side launchers that are laterally spaced from one another in a direction generally perpendicular to the direction of travel of the carriers through the chamber.

Additionally, or in the alternative, the microwave heating system may also include at least two launchers positioned on opposite sides of the microwave chamber as shown by upper and lower sets of microwave launchers 332a and 332b illustrated in FIG. 5. These opposed or oppositely disposed launchers may be oppositely facing, such that launch openings of the launchers are substantially aligned, as shown in FIG. 5, or staggered such that the launch openings of opposed launchers are axially and/or laterally spaced from each other. Several specific launchers and various configurations of multiple launchers suitable for use in the microwave heating zone of the present invention are described in further detail in the '516 application.

Any suitable type of microwave launcher may be used in the microwave heating zone. In some cases, one or more microwave launchers utilized in the microwave heating zone may be tilted at a launch tilt angle of at least 2, at least about 4, at least about 6° and/or not more than about 15, not more than about 10, not more than about 8, or not more than about 6°, as described in detail in the '516 application. Additionally, or in the alternatively, at least one launch opening may be at least partially covered with a microwave-transparent window, as also described in detail in the '516 application. Specific examples of suitable launcher configurations, including particular dimensions, shapes, and orientations, are also described in the '516 application.

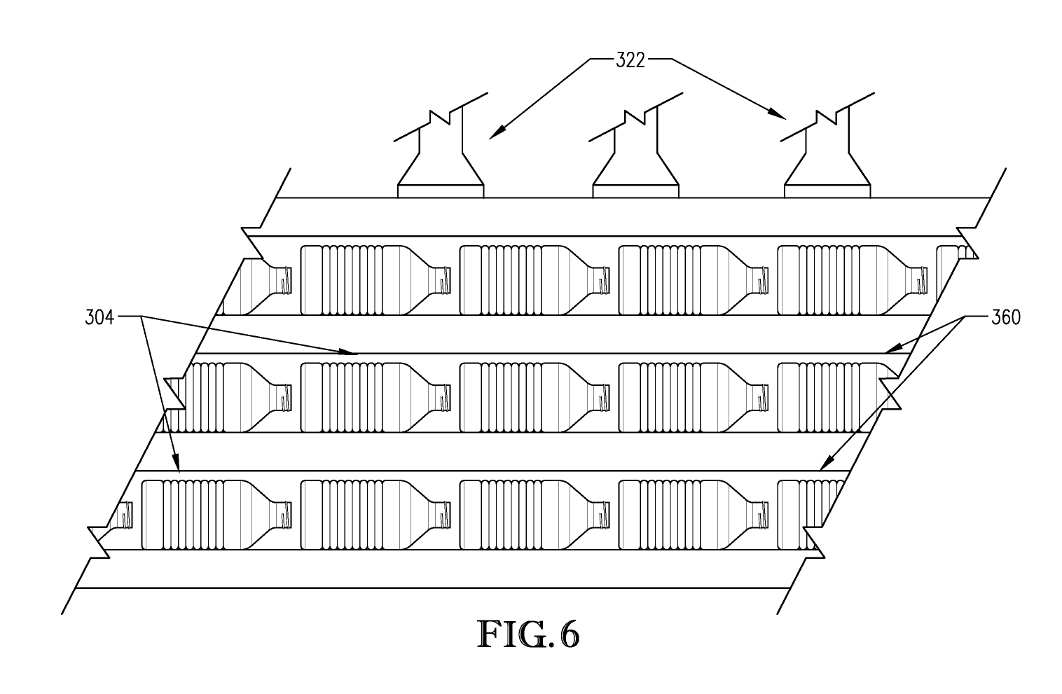

As generally shown in FIG. 5, in some embodiments, microwave chamber 330 may be a single chamber through which a plurality of bottles or other containers may pass during the microwave heating step. In other embodiments, the microwave heating zone may include a plurality of tunnels 360, as shown in FIG. 6. When the microwave heating zone includes tunnels, it can include at least 2, at least 3, at least 4, at least 5, at least 6, at least 7, or at least 8 or more tunnels and/or not more than 15, not more than 14, not more than 12, not more than 10, not more than 8, or not more than 6 tunnels arranged in parallel, as generally shown in FIG. 6. It may also include a single tunnel. The bottles 304 (or other containers) may move through the tunnels 360 via a pump or by a mechanical device (not shown) designed to push the bottles 304 through the individual tunnels 360.

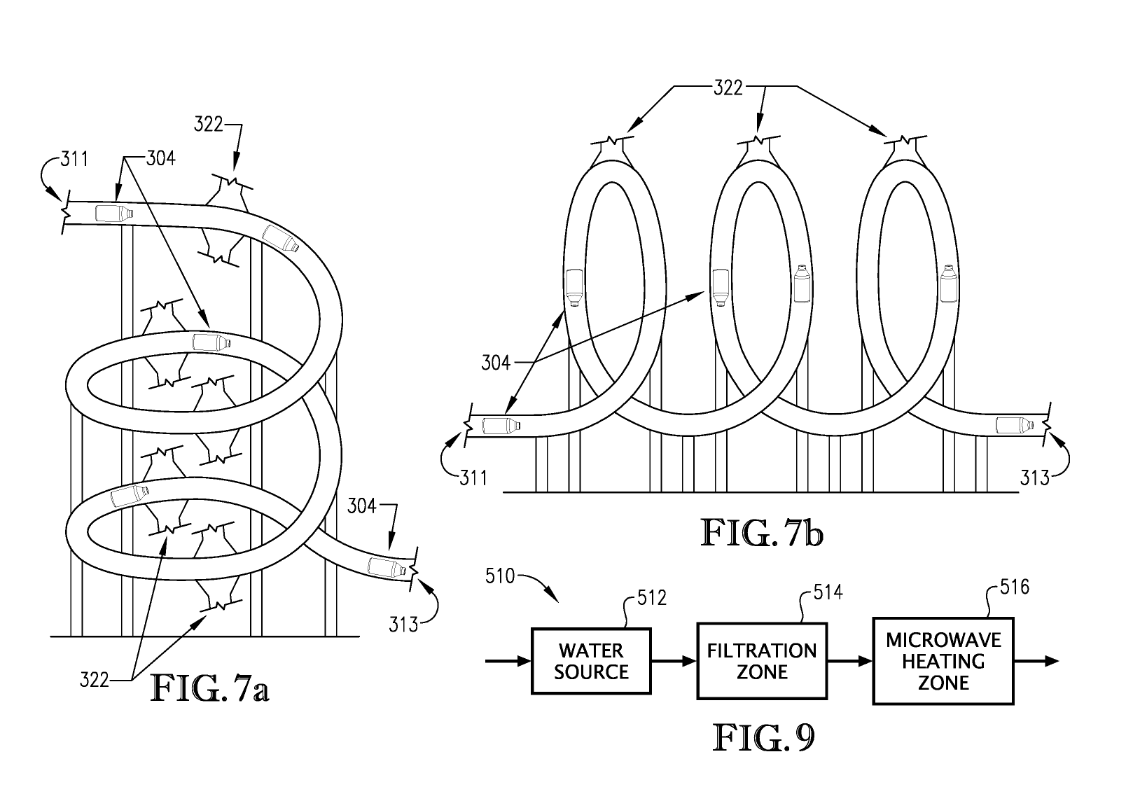

In still other embodiments, the microwave heating chamber 330 may be or comprise a spiral chamber or include at least one spiral section. For example, when microwave heating chamber 330 is a spiral chamber, it may include at least 1, at least 2, at least 3, at least 4, at least 5, or at least 6 bends or curves having an angle greater than 90°. Examples of such microwave heating chambers 330 are shown in FIGS. 7a and 7b. In some cases, the inlet 311 of the microwave chamber may be substantially higher than its outlet 313, thereby forming a vertical spiral as shown in FIG. 7a. Alternatively, the inlet 311 of the microwave chamber may be at nearly the same, or a slightly higher or lower, vertical elevation than its outlet 313, thereby forming a horizontal chamber as shown in FIG. 7b. Use of a spiral-shaped or spiral-containing microwave heating chamber 330 may help reduce the footprint of the microwave heating system and may be useful for retrofitted systems having limited space.

Referring again to FIGS. 1a and 1b, in some applications, at least one agitation device may be included in the thermalization zone 14 and/or microwave heating zone 16 in order to enhance the degree of heat transferred to the containers. Such agitation devices may be used when the thermalization zone 14 and/or microwave heating zone 16 are at least partially filled with a liquid medium. Suitable agitation devices can include, but are not limited to, fluid jets or nozzles, ultrasonic pulses, sonic or acoustic pulses or devices, as well as rifling located along the inside wall of the heating chamber. Alternatively, or in addition, the thermalization zone 14 and/or microwave heating zone 16 may include at least one device to shake, spin, or otherwise disrupt the liquid or semi-liquid material inside the container, thereby enhancing uniformity of heating as well as heat transfer rate. In some applications, the total temperature change of the bulk liquid across the microwave heating zone 16 can be at least about 2, at least about 5, at least about 10, at least about 15, at least about 20, or at least about 25° C. and/or not more than about 50, not more than about 45, not more than about 40, not more than about 35, or not more than about 30° C.

Upon introduction into the microwave heating zone 16, the containers may be passed through the microwave heating chamber while microwave energy is continuously discharged into the chamber. As discussed above, the heating chamber may be at least partially filled with a liquid medium so that the bottles or other containers are submerged in and move through the liquid medium as they are passed through the chamber. In some applications, the microwave chamber can be at least 50, at least about 60, at least about 65, at least about 70, at least about 75, at least about 80, at least about 85, at least about 90, or at least about 95 percent filled with a liquid medium. The liquid medium can include or be water, and it may have a dielectric constant similar to the liquid or semi-liquid material being heated. At least a portion of the microwave energy discharged into the heating chamber may be used to heat the containers so that the coldest region of the liquid or semi-liquid material achieves a temperature at or above a target temperature for a predetermined period of time.

The period of time during which the coldest region of the liquid or semi-liquid material is maintained at or above the target temperature can be any time suitable to achieve the desired kill rate and/or cook time of the liquid or semi-liquid material. In some cases, this can be, for example, 30 seconds to 10 minutes, 45 seconds to 8 minutes, 1 minute to 6 minutes, with the specific time depending, at least in part, on the target temperature. The time may be at least about 30 seconds, at least about 45 seconds, at least about 1 minute, at least about 2 minutes, or at least about 5 minutes and/or not more than about 15 minutes, not more than about 10, not more than about 8, not more than about 6, or not more than about 4 minutes. The target temperature could be a minimum pasteurization temperature of, for example, at least about 70, at least about 75, or at least about 80° C., or it may be a minimum sterilization temperature of, for example, at least about 115, at least about 117, at least about 120, or at least about 121° C. The target temperature may also be within one or more of the ranges discussed previously. When the bottle or other container is at least partially formed from a polymeric material having a glass transition temperature, the target temperature to which the liquid or semi-liquid material is heated may be greater than or less than the glass transition temperature by, for example, at least about 2, at least about 5, at least about 8, at least about 10, at least about 12, or at least about 15° C.

As the bottles or other containers move through the microwave heating chamber 330, they pass by at least one launch opening defined by one or more microwave launchers. As the container passes near a launch opening, the temperature of at least a portion of the liquid or semi-liquid material in the container may increase rapidly to a temperature at or near the target temperature. When the container is at least partially formed from a polymeric material, the temperature of at least a portion of the liquid or semi-liquid material may increase to a temperature at or above the glass transition temperature of the polymer. At the same time, the average temperature of the liquid medium surrounding the container and, in particular, at the wall of the container may be at a temperature lower than the glass transition temperature of the polymer.

As the container moves away from the launch opening, the temperature of its contents may drop, and, in some cases, at least a portion of the liquid or semi-liquid material may drop to a temperature below the target temperature. When the container is formed from a polymer, the temperature of the liquid or semi-liquid material at the wall may drop below the glass transition temperature of the container. The container may be passed by any number of microwave launchers required to achieve a time/temperature combination sufficient for the coldest portion of the liquid or semi-liquid material to achieve a desired kill rate or cook time. However, because the container is only being exposed to elevated temperatures (e.g., near or above the glass transition temperature of the polymeric material) for relatively short periods of time, the container does not deform or rupture. As a result, it has been found that thinner polymeric containers may be used in some embodiments of the present invention while still achieving pasteurization or sterilization results equal to or better than conventional processes.

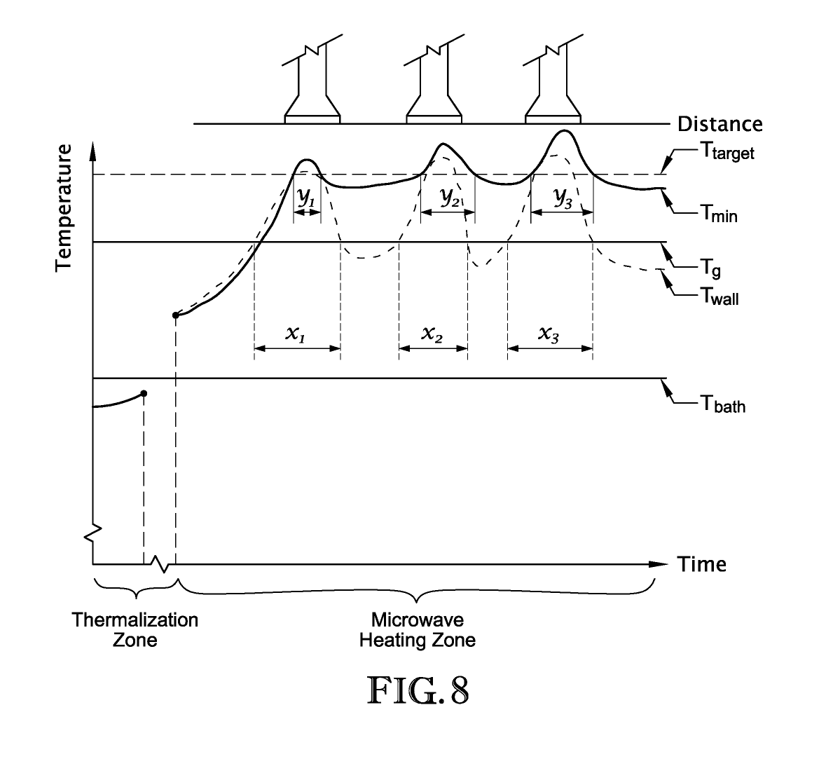

Turning now to FIG. 8 a graph showing the temperature of a liquid or semi-liquid material heated according to embodiments of the present invention is shown as a function of time and position along a microwave heating chamber. The exemplary graph provided in FIG. 8 assumes the container is at least partially formed from a polymeric material having a glass transition temperature (T_{g}), which is less than the target temperature to be achieved during the heating step (T_{target}). FIG. 8 also assumes that the container is surrounded by a fluid having an average temperature (T_{bath}) that is lower than both the target temperature and glass transition temperature of the polymer used to form the container. The fluid used to surround the container may be a liquid medium and can comprise or be water. It should be understood that similar temperature profiles may also be achieved in other embodiments such as, for example, when the container is not formed from a polymeric material (i.e., no T_{g}) or when the target temperature for the liquid or semi-liquid material is less than the glass transition temperature (i.e., position of T_{g} and T_{target} lines switched).

As shown in FIG. 8, when the container passes through the microwave heating zone, the temperature of the coldest portion of the liquid or semi-liquid material in the container (shown as Tₘᵢₙ) increases, as does the temperature of the liquid or semi-liquid material at the container wall (shown as T_{wall}). When the container passes near an opening of one of the microwave launchers, the temperature at the coldest portion of the liquid or semi-liquid material may increase to a temperature at or above the target temperature, as shown in FIG. 8. At the same time, the temperature of the liquid or semi-liquid material at the container wall may also increase, first to a temperature near, at, or above the glass transition temperature of the polymer, then to a temperature at or near the target temperature as the container passes by the first launcher. As the container moves away from the first microwave launcher, the temperature of the coldest portion of the liquid or semi-liquid material decreases slightly, possibly to a temperature below the target temperature and above (or below) the glass transition temperature. The temperature of the liquid or semi-liquid material at the wall of the container also drops quickly, often to a temperature below the glass transition temperature due, at least in part, to its proximity to the lower-temperature fluid bath. Similar trends continue as the container passes by subsequent launchers along the length of the microwave heating chamber.

As the container is heated, the liquid or semi-liquid material at the wall of the container may be kept at or above the glass transition temperature for a minimum amount of time, while still achieving a desired degree of pasteurization or sterilization. For example, as shown in FIG. 8, the total amount of time that the temperature of the coldest portion of the liquid or semi-liquid material is at or above the target temperature, referred to as t₁ and as represented in FIG. 8 by the sum of times y₁ through y₃, can be at least about 10 seconds, 30 seconds, 45 seconds, 1 minute, or 2 minutes and/or not more than about 10 minutes, 8 minutes, 6 minutes, 5 minutes, 3 minutes, 2 minutes, 1 minute, or 30 seconds. Individual values for y₁ through y₃ may be the same or different, and can be at least about 0.5 seconds, 1 second, 2 seconds, 5 seconds, 8 seconds, or 10 seconds and/or not more than about 6 minutes, 5 minutes, 4 minutes, 3 minutes, 2 minutes, or 1, minute, or it can be in the range of from 0.5 seconds to 6 minutes, 5 seconds to 3 minutes, or 10 seconds to 2 minutes.

Additionally, the maximum temperature of the liquid or semi-liquid material at the wall of the container may be at or above the glass transition temperature of the polymeric material for a total period of time, referred to as t₂ and represented in FIG. 8 by the sum of times x₁ through x₃, of at least about 0.5 seconds, 1 second, 2 seconds, 5 seconds, 10 seconds, 30 seconds, 45 seconds, 1 minute, or 2 minutes and/or not more than about 10 minutes, 8 minutes, 6 minutes, 5 minutes, 3 minutes, 2 minutes, or 1 minute. Individual values for x₁ through x₃ can be at least about 0.5 seconds, 1 second, 2 seconds, 5 seconds, 10 seconds, or 15 seconds and/or not more than about 6 minutes, 5 minutes, 3 minutes, 2 minutes, 1 minute, or 30 seconds, or it can be in the range of from 0.5 seconds to 6 minutes, 5 seconds to 3 minutes, or 10 seconds to 2 minutes.

In some cases, the values of t₁ and t₂ may be close, meaning that a significant portion of the total time during which the maximum temperature of the liquid or semi-liquid material at the wall of the vessel is above the glass transition temperature overlaps with the time during which the pasteurization or sterilization of the liquid or semi-liquid material takes place. In some cases, the ratio of t₁ to t₂ can be at least about 0.40, at least about 0.45, at least about 0.50, at least about 0.55, at least about 0.60, at least about 0.65, at least about 0.70, at least about 0.75, at least about 0.80, at least about 0.85, at least about 0.90, or at least about 0.95, with higher ratios generally indicating more efficient heating. In some embodiments, convective heat transfer from the warmer portion of the liquid or semi-liquid material in the container (typically located at or near the geometric center of the container) and the cooler liquid or semi-liquid near the container wall may help facilitate more even heating of the container contents in order to achieve the desired degree of pasteurization or sterilization.

In some cases, this convective heat transfer can be facilitated by inclusion of at least one agitation device within the microwave heating chamber. When used, the agitation device may be the same as, or different than, any agitation devices used in the thermalization zone 14. Suitable agitation devices can include, for example, dynamic agitation devices such as fluid jets or nozzles, ultrasonic pulses, sonic or acoustic pulses or devices, or static agitation devices such as rifling located along the inside wall of the heating chamber. Alternatively, or in addition, the microwave heating chamber can include at least one device to shake, spin, or otherwise disrupt the liquid or semi-liquid material inside the container. Use of agitation devices may help enhance the uniformity of heating of the container contents by, for example, increasing the heat transfer rate between the warmer liquid or semi-liquid material at or near the center of the container and the cooler liquid or semi-liquid near the container wall.

In some embodiments, the cooler temperature of the liquid or semi-liquid material at the wall of the container may be due, at least in part, to the lower temperature of the surrounding liquid (T_{bath}). For example, in some embodiments, the liquid medium surrounding the container during the heating step can have an average temperature, measured near the external wall of the container, that is at least about 2, at least about 5, at least about 10, at least about 15° C. and/or not more than about 30, not more than about 25, or not more than about 20° C. cooler than the temperature of the liquid or semi-liquid at the wall of the container. When the average temperature of the surrounding liquid is lower than the temperature of the liquid or semi-liquid material at the wall of the container, this temperature difference may help minimize the amount of time that the container wall is exposed to temperatures exceeding the glass transition temperature of the polymer used to form the container.

As a result, the temperature of the container wall be at its glass transition temperature for a minimal amount of time, or it may not even reach its glass transition temperature during the heating step. This helps prevents deformation or rupturing of the container during heating. In some embodiments, the temperature of the container wall may be at or above its glass transition temperature for less than 30 seconds, less than 20 seconds, less than 15 seconds, less than 10 seconds, less than 5 seconds, or less than 2 seconds total, or not at all, during the entire heating step. Alternatively, or in addition, the temperature of the container wall may be at least about 2, at least about 3, at least about 5, at least about 8, or at least about 10° C. lower than the glass transition temperature for at least about 50 percent, at least about 60 percent, at least about 70 percent, at least about 80 percent, or at least about 90 percent of the heating step.

Although generally shown in FIG. 8 as including single, longitudinally-spaced apart launchers, it should be understood that the microwave heating systems of the present invention may employ one or more pairs of opposed launchers placed on opposite sides of the microwave heating chamber. Each of the microwave launchers may emit between 1 and 30 kW, between 2 and 25 kW, or between 5 and 20 kW of microwave energy into the heating chamber. In some embodiments, each microwave launcher may be configured to emit at least about 5, at least about 7, at least about 10, at least about 15 kW and/or not more than about 50, not more than about 40, not more than about 30, not more than about 25, not more than about 20, or not more than about 17 kW.

When the system includes two or more microwave launchers, each launcher may emit the same amount of energy as one or more other launchers, or at least one launcher may emit a different (e.g., lower or higher) amount of energy, as compared to at least one of the other launchers. Overall, the total amount of energy discharged into the microwave heating chamber can be at least about 25 kW, at least about 30 kW, at least about 35 kW, at least about 40 kW, at least about 45 kW, at least about 50 kW, at least about 55 kW, at least about 60 kW, at least about 65 kW, at least about 70 kW, or at least about 75 kW and/or not more than about 100 kW, not more than about 95 kW, not more than about 90 kW, not more than about 85 kW, not more than about 80 kW, not more than about 75 kW, not more than about 70 kW, or not more than about 65 kW. Further, although shown as including three consecutive launchers, microwave heating systems employing two or fewer microwave launchers or four or more microwave launchers may also be used, and would be expected to exhibit similar temperature profiles.

In addition to reducing the thermal load to which the container is exposed, the use of microwave energy to efficiently heat a liquid or semi-liquid material as described herein may also improve the quality of the final product. For example, even when the container is not formed from a polymeric material (such as, for example, a glass container), heating the container as described above may reduce the thermal history of the liquid or semi-liquid material therein, thereby minimizing overcooking and heat degradation of the final product. This may help retain desirable organoleptic properties such as taste, texture, and color, as well as retain key functionalities when the liquid or semi-liquid material being processed is a medical, pharmaceutical, nutraceutical, or veterinary fluid. Overall, the systems and methods of the present invention reduce overall processing time, while providing products of equivalent or better quality and meeting or exceeding safety standards.

Referring again to FIGS. 1a and 1b, after being withdrawn from the microwave heating zone 16, the containers may optionally be passed to an optional hold zone 18, wherein the temperature of the containers may remain substantially constant. When present, the containers passing through the hold zone 18 may be there for at least about 30 seconds, at least about 1 minute, at least about 2, at least about 5, or at least about 10 minutes and/or not more than about 15, not more than about 12, not more than about 10, or not more than about 8 minutes. The hold zone may or may not be pressurized and, when the microwave heating system includes a convey line, the convey line may or may not extend through the hold zone. The temperature of the liquid in the hold zone 18 can be at least about 60, at least about 65, at least about 70, at least about 75, at least about 80, at least about 85, at least about 90, at least about 95, at least about 100, at least about 105, at least about 110, or at least about 115° C. and/or not more than about 130, not more than about 125, not more than about 122, not more than about 120, not more than about 115, or not more than about 110° C.

In the hold zone 18, the temperature of the coldest part of the contents of the bottles can be held at a temperature at or above a predetermined minimum temperature of at least about 70° C., at least about 75° C., at least about 80° C., at least about 85° C., at least about 90° C., at least about 95° C., at least about 100° C., at least about 105° C., at least about 110° C., at least about 115° C., or at least about 120° C., at least about 121° C., at least about 122° C. and/or not more than about 130° C., not more than about 128° C., or not more than about 126° C. When pressurized, the pressure within the hold zone 18 can be at least about 5, at least about 10, at least about 15, or at least about 20 psig and/or not more than about 60, not more than about 55, not more than about 50, not more than about 45, not more than about 40, not more than about 35, or not more than about 30 psig.

After exiting the hold zone, the containers may be passed to a cooling, or quench zone 20, wherein the bottles are cooled as rapidly as possible via submersion in a cooled fluid. In the quench zone 20, the temperature of the containers may be reduced by at least about 2, at least about 5, at least about 10, at least about 15, at least about 20, at least about 25, at least about 30, at least about 40, or at least about 50° C. and/or not more than about 100° C., not more than about 75° C., or not more than about 50° C. to a temperature of not more than about 50, not more than about 45, not more than about 40, not more than about 35, not more than about 30, or not more than about 25° C.

Any suitable fluid may be used in the cooling zones and, in some cases, the fluid may include a liquid similar to or different than the liquid used in the microwave heating zone and/or the hold section. The temperature of the liquid in the cooling zones can be not more than about 50, not more than about 45, not more than about 40, not more than about 35, not more than about 30, or not more than about 27° C. The cooling zones may be pressurized, unpressurized (e.g., atmospheric), or it may include both pressurized and unpressurized sections. Similarly, if the microwave heating system includes a convey line, the convey line may or may not extend through the cooling zone. Additionally, the cooling zones may be at least partially liquid filled, or it may be under atmospheric conditions. In some applications, the cooling zone may include both a liquid-filled section and an atmospheric section.

As shown in FIGS. 1a and 1b, the quench zone 20 may include a high-pressure cooling zone 24a and a low-pressure cooling zone 24b separated by a transfer zone 15b. The pressure of the high-pressure cooling zone 24a can be at least about 5, at least about 10, at least about 15, or at least about 20 psig and/or not more than about 60, not more than about 55, not more than about 50, not more than about 45, not more than about 40, not more than about 35, or not more than about 30 psig, while the pressure of the low-pressure cooling zone 24b can be not more than about 15, not more than about 10, not more than about 8, not more than about 5, not more than about 3, or not more than about 2 psig. In some cases, the difference in pressure between the high-pressure cooling zone 24a and the low-pressure cooling zone 24b can be at least about 1, at least about 2, at least about 5, at least about 10, at least about 15, or at least about 20 psig and/or not more than about 60, not more than about 55, not more than about 50, not more than about 45, not more than about 40, not more than about 35, or not more than about 30 psig.

One particular application in which microwave heating systems of the present invention may be used is in the sterilization of bottled water. One example of such a system 510 is shown in FIG. 9. As shown in FIG. 9, water originating from a source 512, such as a river, lake, stream, or other suitable source, may be used to fill a plurality of bottles without first being subjected to reverse osmosis, UV treatment, distillation, micron filtration, or ozonation. In some applications, the water may first be introduced into an optional filtration zone 514, wherein it may be filtered to remove dissolved or suspended solids and/or it may be passed through one or more filtration beds including, for example, carbon black for removing one or more other unwanted materials. Then, as shown in FIG. 9, the water may be introduced into a microwave heating system 516, which can be configured similarly to the systems described herein and, in particular, as described with respect to FIGS. 1a and 1b.

In some cases, the substantially untreated water may be subjected to sterilization and/or pasteurization in the microwave heating system 516 as described herein and the resulting bottles may have an overall contaminant level commensurate with bottled water that has undergone extensive processing prior to being bottled. Use of systems and methods of the present invention to prepare sterilized bottled water may help provide clean drinking water to areas of the world where potable water is scarce.

Microwave heating systems of the present invention can be commercial-scale heating systems capable of processing a large volume of containers in a relatively short time. In contrast to conventional retorts and other small-scale systems that utilize microwave energy to heat a plurality of containers, microwave heating systems as described herein can be configured to achieve an overall production rate of at least about 15 packages per minute per convey line, at least about 20 packages per minute per convey line, at least about 25 packages per minute per convey line, or at least about 30 packages per minute per convey line, measured as described in the '516 application.

When the containers include bottles, microwave heating systems of the present invention may have an overall production rate of at least 5, at least about 10, at least about 25, at least about 50, or at least about 100 bottles per minute and/or not more than about 1500, not more than about 1250, not more than about 1000, not more than about 900, not more than about 750, not more than about 500, not more than about 350, not more than about 200, not more than about 150, not more than about 100, or not more than about 75 bottles per minute. Lower production rates may be used for more delicate or specialty items, while higher rates may be used for processing commodity goods.

The preferred forms of the invention described above are to be used as illustration only, and should not be used in a limiting sense to interpret the scope of the present invention. Obvious modifications to the exemplary one embodiment, set forth above, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

What is claimed is:

1. A process for pasteurization or sterilization of liquid or semi-liquid material, said process comprising:
(a) passing a plurality of containers at least partially filled with said liquid or semi-liquid material through a microwave heating chamber at least partially filled with a liquid medium, wherein said containers are at least partially formed from a polymeric material having a glass transition temperature;
(b) discharging microwave energy into said microwave heating chamber via at least one microwave launcher; and
(c) heating said containers using at least a portion of the microwave energy discharged into said microwave heating chamber, wherein said containers are submerged in said liquid medium during said heating, wherein said heating is sufficient to increase the minimum temperature of the coldest region of said liquid or semi-liquid medium to temperature at or above a target temperature for a predetermined amount of time, wherein said target temperature is greater than said glass transition temperature of said polymeric material, and
wherein during at least a portion of said heating, the average temperature of said liquid medium at the wall of each of said containers is below said glass transition temperature of said polymeric material.

2. The process of claim 1, wherein said discharging includes discharging microwave energy into said microwave heating chamber via two or more microwave launchers and said passing includes passing each of said containers by each of said launchers, wherein the temperature of said liquid or semi-liquid material at the wall of each container increases to a temperature greater than said glass transition temperature when the container is passing by one of said launchers and decreases to a temperature less than said glass transition temperature when the container is not passing by one of said launchers, wherein the maximum temperature of said liquid or semi-liquid material at the wall of each of said containers is above said glass transition temperature of said polymeric material for a total time of at least 1 second and not more than 10 minutes.

3. The process of claim 2, wherein each of said launchers discharges at least 5 kW and not more than 25 kW of microwave energy into said microwave heating chamber, and wherein the maximum temperature of said liquid or semi-liquid material at the wall of each of said containers is greater than said glass transition temperature for at least 0.5 seconds and not more than 2 minutes while passing by one of said launchers.

4. The process of claim 1, wherein during said heating, said minimum temperature of the coldest region of said liquid or semi-liquid material in each of said containers is maintained at or above a target temperature for a first period of time, t₁, and wherein during said heating, the maximum temperature of said liquid or semi-liquid material at the wall of the container is maintained at or above said glass transition temperature for a second period of time, t₂, and wherein the ratio of t₁ to t₂ is at least 0.40.

5. The process of claim 1, further comprising during at least a portion of said passing and/or said heating, agitating said bottles with at least one agitation device.

6. The process of claim 1, wherein said target temperature is at least about 75° C. and said glass transition temperature is at least 50° C.

7. The process of claim 1, wherein said target temperature is at least 115° C. and said glass transition temperature is at least 85° C.

8. The process of claim 1, wherein the difference between said target temperature and said glass transition temperature is at least 2° C.

9. The process of claim 1, wherein said polymer is selected from the group consisting of polyethylene terephthalate, polyethylene, polypropylene, polylactic acid, polyhydroxyalkanoates, polybutylene succinate, biopolymers, and combinations thereof, wherein each of said containers has a nominal liquid capacity in the range of from 5 to 50 fluid oz., and wherein said liquid or semi-liquid material comprises a food, a beverage, a medical fluid, a pharmaceutical fluid, a nutraceutical fluid, or a veterinary fluid.

10. The process of claim 1, wherein said liquid or semi-liquid material is selected from the group consisting of tea, juice, mineral drinks, electrolyte drinks, energy drinks, vitamin drinks, shakes, smoothies, dairy drinks, alcoholic drinks, and coffee drinks.

11. The process of claim 1, wherein said liquid or semi-liquid material is water.

12. The process of claim 1, wherein said liquid or semi-liquid material comprises solid particulates having an average particle size in the range of from 1 to 25 mm.

13. The process of claim 1, wherein said liquid or semi-liquid material is selected from the group consisting of soups, jams, jellies, syrups, salsas, creams, sauces, and condiments.

14. A process for pasteurization or sterilization of liquid or semi-liquid material, said process comprising:
(a) at least partially filling a plurality of containers with said liquid or semi-liquid material, wherein the maximum temperature of said liquid or semi-liquid material during said filling is in the range of from 110° F. to a first target temperature;
(b) passing said containers through a microwave heating zone on a convey line;
(c) continuously directing microwave energy toward said containers passing through said microwave heating zone on said convey line; and
(d) heating said containers to a second temperature that is greater than said first target temperature using at least a portion of said microwave energy in order to pasteurize or sterilize said liquid or semi-liquid material within each of said containers.

15. The process of claim 14, wherein said containers are formed from a polymeric material having a glass transition temperature, wherein said first target temperature in step (a) is less than said glass transition temperature and said second temperature in step (d) is greater than said glass transition temperature.

16. The process of claim 15, wherein said target temperature is at least 5° F. cooler than said glass transition temperature.

17. The process of claim 15, wherein said microwave heating zone includes a liquid-filled microwave heating chamber, wherein said containers are submerged in a liquid medium during said passing and said heating, wherein said heating is sufficient to increase the minimum temperature of the coldest region of said liquid or semi-liquid material to a temperature at or above a target temperature for a predetermined amount of time, wherein said target temperature is greater than said glass transition temperature, wherein during at least a portion of said heating, the average temperature of said liquid medium at the wall of said containers is below said glass transition temperature, and wherein during said heating, the maximum temperature of said liquid or semi-liquid material at the wall of each of said containers is above said glass transition temperature of said polymeric material for a total time of at least 0.5 seconds and not more than 3 minutes.

18. The process of claim 14, wherein said target temperature is less than 185° F.

19. The process of claim 14, wherein said containers comprise bottles, wherein said passing comprises passing a plurality of said bottles through at least one heating tunnel in a single-file line, and wherein said bottles are arranged in a side-by-side configuration or an end-to-end configuration in said heating tunnel.

20. The process of claim 19, wherein said bottles are in an upright position in said heating tunnel.

21. The process of claim 19, wherein said bottles are in an inverted position during at least a portion of said passing.

22. The process of claim 14, further comprising prior to said heating, thermalizing the filled containers in a thermalization zone located upstream of said microwave heating zone, wherein at least about 85 percent of said containers withdrawn from said thermalization zone have a temperature within about 5° C. of one another.

23. The process of claim 14, wherein said liquid or semi-liquid material introduced into said containers during said filling has not been pasteurized or sterilized, and further comprising prior to said passing, sealing each of said containers with a sealing device, wherein neither said container nor said sealing device has been pasteurized or sterilized prior to said filling and said sealing.

24. The process of claim 14, wherein said containers are formed from glass.

25. The process of claim 14, wherein said containers are formed from a polymeric material selected from the group consisting of polyethylene terephthalate, polyethylene, polypropylene, polylactic acid, polyhydroxyalkanoates, polybutylene succinate, biopolymers, and combinations thereof.

26. The process of claim 14, wherein said liquid or semi-liquid material is selected from the group consisting of tea, juice, mineral drinks, electrolyte drinks, energy drinks, vitamin drinks, shakes, smoothies, dairy drinks, alcoholic drinks, and coffee drinks.

27. The process of claim 14, wherein said liquid or semi-liquid material is selected from the group consisting of soups, jams, jellies, syrups, salsas, creams, sauces, and condiments.

28. A microwave heating system for heating a plurality of containers filled with a liquid or semi-liquid material, said system comprising:
at least one microwave generator for generating microwave energy;
a microwave heating chamber for heating said containers, wherein said microwave heating chamber has an inlet and an outlet; and
at least two spaced apart microwave launchers for emitting at least a portion of said microwave energy into said microwave heating chamber, wherein at least a portion of said microwave energy is used to heat said containers,
wherein said inlet to said microwave heating chamber is at a higher vertical elevation than the outlet of said microwave heating chamber.

29. The system of claim 28, wherein at least a portion of said microwave heating chamber is configured to move said containers through said microwave heating chamber by gravity, wherein said microwave heating chamber includes at least one bend so that the direction of travel of said container through said bend has an angle of at least 90°, wherein said microwave heating chamber comprises at least one heating tunnel configured to permit a line of said containers to pass therethrough in a direction of travel extending through said microwave heating chamber, and wherein said containers are arranged in a single-file line in a side-by-side or an end-to-end configuration in said heating tunnel, and wherein the ratio of the height of said heating tunnel to the dimension of each of said containers perpendicular to said direction of travel is less than 1.5:1.

30. The system of claim 28, wherein said microwave heating chamber is at least partially filled with a liquid medium and wherein said containers are configured to pass through said liquid medium within said microwave heating chamber, wherein said system is configured to heat containers of at least two different sizes, and wherein said system has an overall production rate in the range of from 5 to 900 containers per minute.
